(19) United States
(12) Patent Application Publication
(10) Pub. No.: US 2010/0099885 A1
(43) Pub. Date: Apr. 22, 2010

(54) PROCESS FOR THE PREPARATION OF FORM I AND FORM II OF RITONAVIR

(76) Inventors: Mukesh Kumar Sharma, Gurgaon (IN); Chandra Has Khanduri, Gurgaon (IN); Pankaj Kumar Singh, Kanpur (IN); Yoginder Pal Sachdeva, Abohar (IN)

Correspondence Address: Ranbaxy Inc., Intellectual Property Department, 600 College Road East, PRINCETON, NJ, 08540, US

(21) Appl. No.: 12/443,749

(22) PCT Filed: Oct. 1, 2007
(86) PCT No.: PCT/IB2007/053988

§ 371 Date: Dec. 11, 2009

(30) Foreign Application Priority Data
Oct. 3, 2006 (IN) 2173/DEL/2006

(51) Int. Cl. C07D 277/30

(52) U.S. Cl. 548/204

(57) ABSTRACT

The present invention relates to processes for the preparation of Form I and Form II of Ritonavir, wherein no seed crystals are used.

### FIELD OF THE INVENTION

The present invention relates to processes for the preparation of Form I and Form II of Ritonavir.

### BACKGROUND OF THE INVENTION

Ritonavir of Formula I is chemically, [5S-(5R*,8R*,10R*,11R*)]-10-Hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester and is indicated in combination with other anti-retroviral agents for the treatment of HIV-infection.

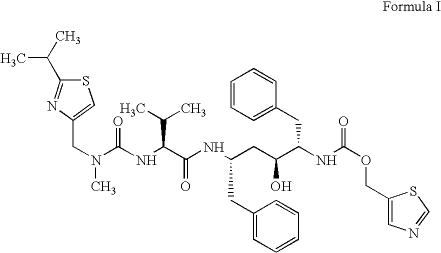

U.S. Pat. No. 5,541,206 (the '206 patent) and its equivalent PCT Patent Application WO 94/14436, and U.S. Pat. No 5,567,823 (the '823 patent) discloses a process for the preparation of Ritonavir.

PCT Patent Application WO 00/04016 (the '016 application) provides processes for preparation of crystalline Form 11 and amorphous Ritonavir. The disclosure of the '016 application suggests that the product obtained as per the processes of the '206 patent and the '823 'patent is Form I of ritonavir. That disclosure provides a process for the preparation of Form I of Ritonavir from Form II of Ritonavir wherein seed crystals of Form I of Ritonavir are used. That disclosure also provides a process for the preparation of Form II of Ritonavir from Form I of Ritonavir or a mixture of Form I and Form II of Ritonavir wherein the process involves the use of seed crystals of (2S)-N-((1S)-1-Benzyl-2-((4S,5S)-4-benzyl-2-oxo-1-3-oxazolidin-5-yl)ethyl)-2-((((2-isopropyl-1,3-thiazol-4-yl)methyl)amino)carbonyl)amino)-3-methylbutanamide or Form II of Ritonavir.

U.S. Patent Application US 2004/0024031 describes Form III, Form IV and Form V of Ritonavir, and provides processes for their preparation. It also provides a process for the preparation of Form I of Ritonavir.

Org. Proc. Res. & .Dev., (2000), 4, 413-417 provides a process for the preparation of Form I of Ritonavir contaminated with less than 3% Form II of Ritonavir, wherein seed crystals of Form I of Ritonavir are used. It also provides a process for the preparation of Form II of Ritonavir wherein seed crystals of Form II of Ritonavir are used.

### SUMMARY OF THE INVENTION

The present inventors have found a simple process for the preparation of Form I and Form II of Ritonavir, wherein no seed crystals are used.

As part of the present invention the term “Form I of Ritonavir” refers to Form I of ritonavir having less than 5% of Form II of ritonavir as determined by X-Ray Powder Diffraction (XRPD).

### DETAILED DESCRIPTION OF THE INVENTION

A first aspect provides a process for the preparation of Form I of Ritonavir, comprising of:
a) dissolving ritonavir in an organic solvent;
b) contacting the solution obtained in step a) with an anti-solvent; and
c) isolating Form I of Ritonavir from the reaction mixture thereof;
   wherein no seed crystals are used.

Ritonavir can be prepared by processes known to skilled artisan. The so-obtained Ritonavir is dissolved in an organic solvent, such as esters, lower alkanols, ethers, ketones, polar aprotic solvents, halogenated hydrocarbons or mixtures thereof in some embodiments the organic solvent can be an ester, for example, ethyl acetate. The mixture so obtained is contacted with an anti-solvent wherein the anti-solvent can be C₅₋₇ straight or branched chain alkanes, C₅₋₇ cycloalkanes, C₄₋₁₂ ethers, petroleum ether or mixtures thereof is added. In some embodiments the anti-solvent can be an alkane, for example, n-heptane. The addition can be completed in, for example, about 1-5 minutes, while maintaining the temperature at, for example, about 0-30° C. The reaction mass can be stirred, filtered and dried to get Form I of Ritonavir.

A second aspect provides a process for the preparation of Form II of Ritonavir, comprising:
a) dissolving ritonavir in an organic solvent;
b) contacting an anti-solvent with the solution obtained in step a); and
c) isolating Form II of Ritonavir from the reaction mixture thereof,
   wherein no seed crystals are used.

Ritonavir can be prepared by processes known to skilled artisans. The so-obtained Ritonavir is dissolved in an organic solvent such as esters, lower alkanols, ethers, ketones, polar aprotic solvents, halogenated hydrocarbons or mixtures thereof. In some embodiments, the organic solvent can be an ester, for example, ethyl acetate. An anti-solvent such as C₅₋₇ straight or branched chain alkanes, C₅₋₇ cycloalkanes, C₄₋₁₂ ethers, petroleum ether or mixtures thereof, is contacted with the solution so obtained. In some embodiments, the anti-solvent can be an alkane, such as n-heptane. The addition can be completed in for example, about 30-40 minutes, while maintaining the temperature at for example, about 40-50° C. The such as reaction mass is stirred, filtered and dried to get Form II of Ritonavir.

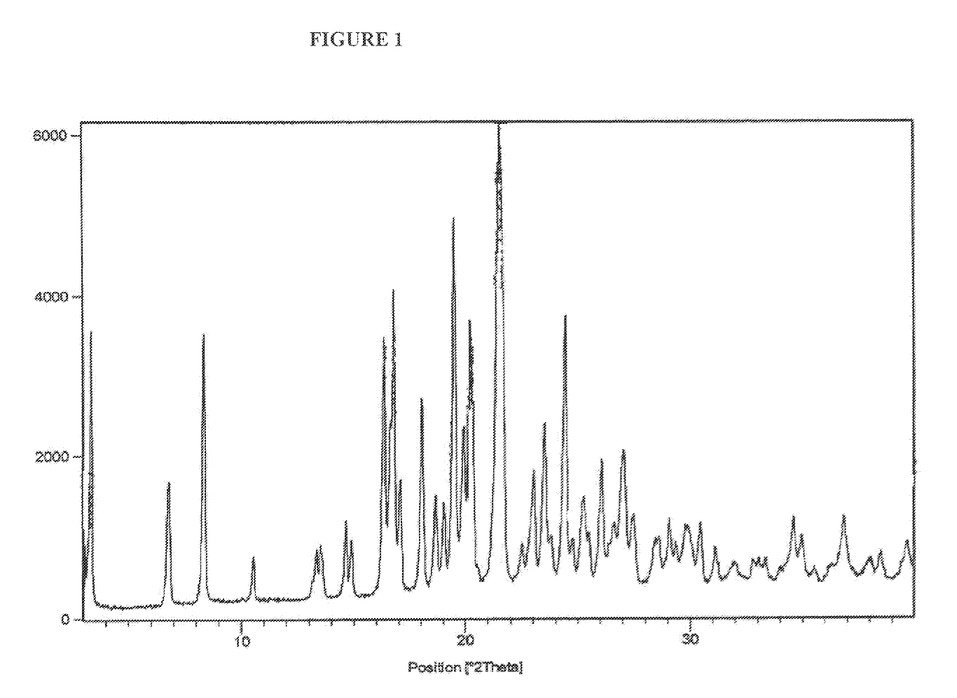

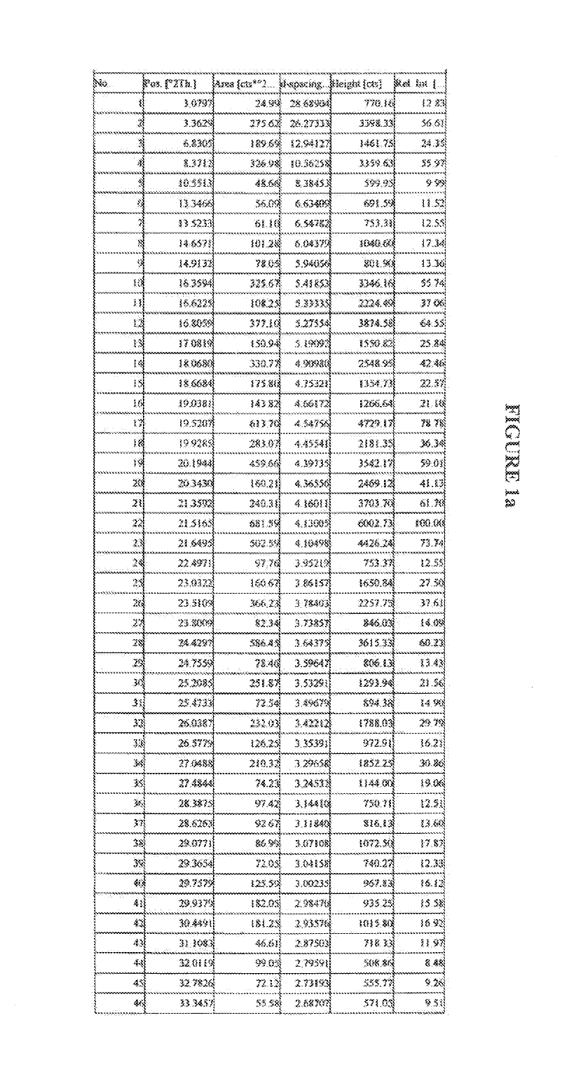

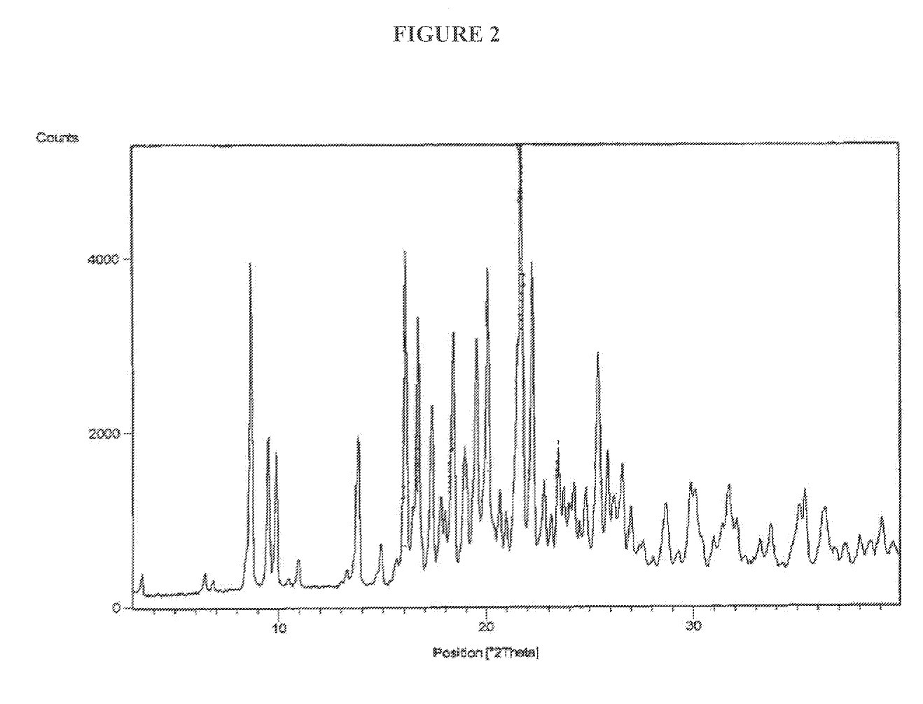

FIG. 1 is an XRD (X-Ray powder Diffraction) spectrum of Form I of Ritonavir.

FIG. 1a is a table listing the positions and intensities of spectral features found in FIG. 1.

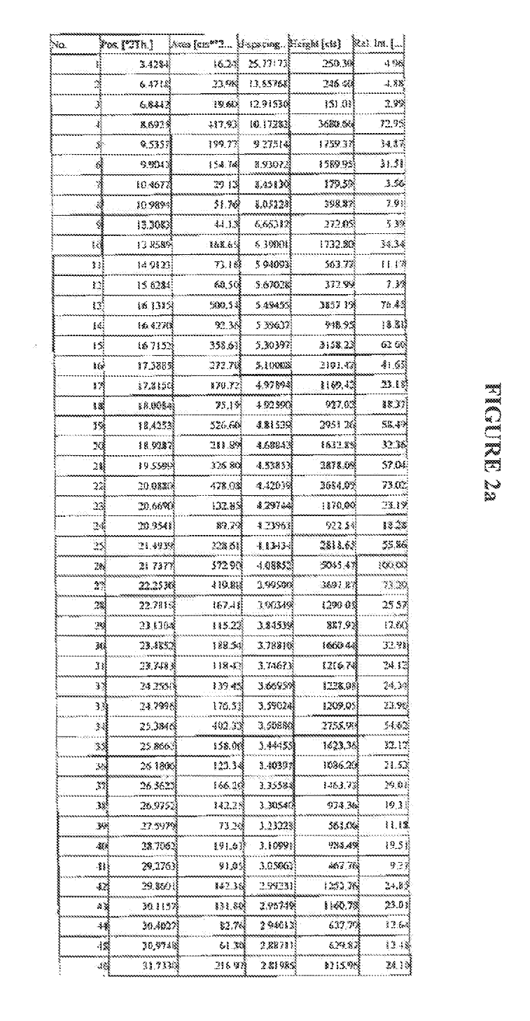

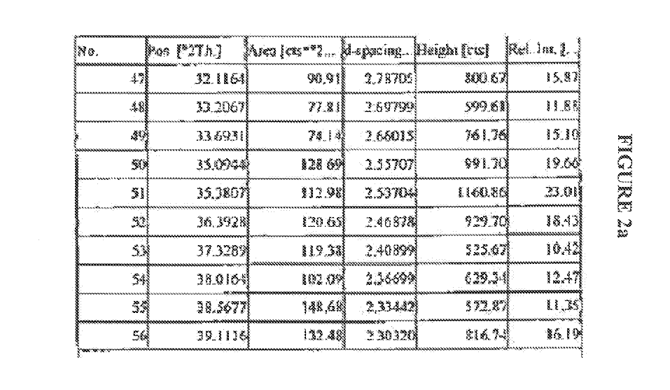

FIG. 2 is an XRD (X-Ray powder Diffraction) spectrum of Form II of Ritonavir.

FIG. 2a is a table listing the positions and intensities of spectral features found in FIG. 2.

While the present invention has been described in terms of its specific embodiments, certain modifications and equivalents will be apparent to those skilled in the art and are intended to be included within the scope of the present invention.

### EXAMPLES

### Example 1

### Preparation of Form I of Ritonavir

Ritonavir (100 g) was suspended in ethyl acetate (400 ml). The mixture was heated at 70° C. and stirred to get a clear solution. The solution was cooled to 50° C. Activated charcoal (5 g) was added to the solution, stirred for 15 minutes at 55-50° C. and filtered through hyflobed. The hyflobed was washed with ethyl acetate (50 ml) and the filtrate was collected, The filtrate was charged into n-Heptane (1000 ml) at 20-25° C. in 2-3 minutes, maintaining the temperature at 20-25° C. and stirred for 4 hours at 20-25° C. The solid obtained was filtered at 20-25° C. and washed with n-heptane (100 ml) by making slurry at 20-25° C. and dried to obtain the title compound, in a yield of 95 g.

### Example 2

### Preparation of Form II of Ritonavir

Ritonavir (100 g) was suspended in ethyl acetate (400 ml). The mixture was heated at 70° C. and stirred to get a clear solution. The solution was cooled to 50° C. Activated charcoal (5 g) was added to the solution, stirred for 15 minutes at 55-50° C. and filtered through hyflobed. The hyflobed was washed with ethyl acetate (50 ml) and the filtrate was collected. n-Heptane (1000 ml) was charged into the filtrate at 40-50° C. in 30-40 minutes, stirred at the same temperature for 1 hour, cooled to 20-25° C. and stirred for 4 hours at 20-25° C. The solid obtained was filtered at 20-25° C. and washed with n-heptane (100 ml) by making slurry at 20-25° C. and dried to obtain the title compound, in yield 95 g.

We claim:

1. A process for the preparation of Form I of Ritonavir, comprising:
a) dissolving ritonavir in an organic solvent;
b) contacting solution obtained in step a) with an anti-solvent; and
c) isolating Form I of Ritonavir from the reaction mixture thereof,
wherein no seed crystals are used.

2. The process according to claim 1, wherein the solution obtained in step a) is not concentrated.

3. A process according to claim 1, wherein the solution obtained in step a) is contact with an anti-solvent, for about 2-3 minutes, while maintaining the temperature at about 20-25° C.

4. A process according to claim 1, wherein ritonavir is dissolved in organic solvent selected from esters, lower alkanols, ethers, ketones, polar aprotic solvents, halogenated hydrocarbons or mixtures thereof.

5. A process according to claim 4, wherein the organic solvent is ethyl acetate.

6. A process according to claim 1, wherein the anti-solvent is selected from C₅₋₇ straight or branched chain alkane, C₅₋₇ cycloalkane, C₄₋₁₂ ethers, petroleum ether or mixtures thereof.

7. A process according to claim 6, wherein the anti-solvent is n-heptane.

8. A process for the preparation of Form II of Ritonavir, comprising:
a) dissolving ritonavir in an organic solvent,
b) contacting an anti-solvent with the solution obtained in step a); and
c) isolating Form II of Ritonavir from the reaction mixture thereof,
wherein no seed crystals are used.

9. A process according to claim 8, wherein an anti-solvent is contacted with to the solution obtained in step a), for about 30-40 minutes, while maintaining the temperature at about 40-50° C.

10. A process according to claim 8, wherein ritonavir is dissolved in organic solvent selected from esters, lower alkanols, ethers, ketones, polar aprotic solvents, halogenated hydrocarbons or mixtures thereof.

11. A process according to claim 10, wherein the organic solvent is ethyl acetate.

12. A process according to claim 8, wherein anti-solvent is selected from C₅₋₇ straight or branched chain alkane, C₅₋₇ cycloalkane, C₄₋₁₂ ethers, petroleum ether or mixtures thereof.

13. A process according to claim 12, wherein the anti-solvent is n-heptane.
